(19) United States
(12) Patent Application Publication
(10) Pub. No.: US 2005/0079234 A1
(43) Pub. Date: Apr. 14, 2005

(54) Compositions comprising herbs and method for immunomodulation

(76) Inventor: Yang Chi Chiang, Taichung (TW)

Correspondence Address: HARNESS, DICKEY & PIERCE, P.L.C., P.O. BOX 8910, RESTON, VA, 20195, US

(21) Appl. No.: 10/927,498

(22) Filed: Aug. 27, 2004

Related U.S. Application Data
(60) Provisional application No. 60/497,929, filed on Aug. 27, 2003.

(51) Int. Cl. A61K 35/78

(52) U.S. Cl. 424/728; 424/729; 424/773

(57) ABSTRACT

The present invention provides a composition for use in immunomodulation comprising licorice root, extracts of American ginseng, Radix Paeoniae alba and green tea (Camellia sinensis). The invention further provides a method for the treatment of diseases caused or affected by malfunction of immune system.

### FIELD OF THE INVENTION

The invention relates to compositions comprising herbs and methods for immunomodulation.

### DESCRIPTION OF THE PRIOR ART

Yi-fey Ruenn-hou (YR) Tea is an old prescription used for centuries in the traditional Chinese medicine for treating tonsillitis. 10% licorice root, 10% American ginseng, 10% white peony root and 70% green tea composed the recipe. Licorice roots (Glycyrrhiza uralensis), American ginseng (Panax quinquefolius), and white peony root (Radix paeoniae alba, RPA) are three widely used valuable traditional Chinese medicines. Green tea (Camellia sinensis) is a popular consumed beverage.

As the oldest and most frequently employed botanicals in Chinese medicine, licorice contains triterpenoids, such as glycyrrhizin and its aglycone glycyrrhizic acid, various polyphenols, and polysaccharides. A number of pharmaceutical effects of licorice, e.g. anti-inflammatory, antiviral, antiulcer, and anticarcinogenesis, are known or suspected.

The main constituent of American Ginseng is ginsenoside, which can combat stress, enhance both the central nervous and immune systems and contribute toward maintaining optimal oxidative status against certain chronic disease states and aging (Kitts, D.; Hu, C. Efficacy and safety of ginseng. Public Health Nutr 2000, 3:473-485). Radix paeoniae alba is the root of a traditional Chinese herb named Paeonia lactiflora pallas, which is commonly used to treat liver diseases in China for centuries. Several earlier studies have indicated that Radix paeoniae (RP) has anticancer growth activity.

Epicatechin derivatives, which are commonly called ‘polyphenols’, are the active ingredients in green tea and possess antioxidant, anti-inflammatory and anti-carcinogenic properties. Green tea polyphenol has potent anti-oxidative activity and anti-inflammatory effects by decreasing cytokine production (Matsuoka, K.; Isowa, N.; Yoshimura, T.; Liu, M.; Wada, H. Green tea polyphenol blocks H(2)O(2)-induced interleukin-8 production from human alveolar epithelial cells. Cytokine 2002, 18: 266-273). Craig (Craig, W. J. Health-promoting properties of common herbs. Am J Clin Nutr 1999, 70: 491S-499S) found that natural antioxidants or phenolic compounds rich in plant foods such as fruits and herbs, and terpenoids would provide a milieu of phytochemicals, non-nutritive substances in plants that possess health-protective benefits. Pharmacological experiments have preliminarily demonstrated that the compound prescription is better than its individual components in the range and potency of pharmacological activities (Huang, L.; Liu, J.; Li, D.; Wang, Z.; Ye, W.; Cai, B.; Liu, M.; Li, M. A study on components and compound prescription of huangqin decoction Zhongguo Zhong Yao Za Zhi 1991, 16: 177-181).

A study demonstrated that Ginseng radix and Radix paeoniae alba in a phylopharmaceutical that augmented monocyte and leucocyte counts in the peripheral blood (Son, C. G.; Han, S. H.; Cho, J. H.; Shin, J. W.; Cho, C. H.; Lee, Y. W.; Cho, C. K. Induction of hemopoiesis by Saenghyuldan, a mixture of Ginseng Radix, Paeoniae Radix Alba, and Hominis Placenta extracts. Acta Pharmacol Sin 2003, 24: 120-126).

The mechanism underlying these activities is unclear and remains to be elucidated. However, as a pharmaceutical composition constituted by licorice root, American ginseng, Radix paeoniae alba and green tea, its effect on T-helper (TH) cytokine modulation in vivo has not been investigated.

### BRIEF DESCRIPTION OF THE INVENTION

The present invention provides a composition for use in immunomodulation comprising licorice root, extracts of American ginseng, Radix Paeoniae alba and green tea (Camellia sinensis).

The present invention further provides a method for the treatment of diseases caused or affected by malfunction of the immune system, which comprises the administration to a human or animal patient in need thereof the composition of the invention.

### BRIEF DESCRIPTION OF THE DRAWING

FIG. 1. The ratio of cytokines in mice given 2 mg/ml (group B) YR Tea compared with those given water (group A) from months 1 to 6.

FIG. 2. The ratio of cytokines in mice given 8 mg/ml (group C) YR Tea compared with those given water (group A) from months 1 to 6.

FIG. 3. The ratio of cytokines in mice given 40 mg/ml (group D) YR Tea compared with those given water (group A) from months 1 to 6.

### DETAILED DESCRIPTION OF THE INVENTION

The present invention provides a composition for use in immunomodulation comprising 1 to 30 weight percent of licorice root, 1 to 30 weight percent of American ginseng, 1 to 30 weight percent of Radix Paeoniae alba and 3 to 97 weight percent of green tea (Camellia sinensis).

In the composition of the invention, the concentration of licorice root ranges from 1 to 25 weight percent., preferably 20, more preferably 15 and the most preferably 10 weight percent.

In the composition of the invention, the concentration of American ginseng ranges from 1 to 25 weight percent., preferably 20, more preferably 15 and the most preferably 10 weight percent.

In the composition of the invention, the concentration of Radix Paeoniae alba ranges from 1 to 25 weight percent., preferably 20, more preferably 15 and the most preferably 10 weight percent.

In the composition of the invention, the concentration of green tea ranges from 3 to 97 weight percent, preferably 40-95, more preferably 50-80 and the most preferably 70 weight percent.

In the components of the composition, the components could be in the form of extract (such as licorice root extract, American ginseng extract, Radix Paeoniae alba extract and green tea extract). The preferred extract of the components is water extract.

The pharmaceutically, veterinarily or dietetically acceptable excipient may be a solvent (such as water), dispersion medium, coating, isotonic or absorption delaying agent, sweetener or the like. These include any and all solvents, dispersion media, coatings, isotonic and absorption delaying agents, sweeteners and the like. These pharmaceutically acceptable carriers may be prepared from a wide range of materials including, but not limited to, diluents, binders and adhesives, lubricants, disintegrants, coloring agents, bulking agents, flavoring agents, sweetening agents and miscellaneous materials such as buffers and adsorbents that may be needed in order to prepare a particular dosage form. The use of such media and agents for pharmaceutically active substances is well known in the art. Except insofar as any conventional media or agent is incompatible with the active ingredient, its use in the present compositions is contemplated.

The present composition can be provided in any convenient form and for administration by any route such as orally, topically or parenterally. It can be provided as a dietary supplement or any pharmaceutical dosage form. It can be formulated into a food or drink, and provided, for example, as a snack bar; a cereal, a drink, a gum, or in any other easily ingested form.

The term “Immunomodulation” used in the invention is a developing segment of immunopharmacology. Immunomodulator compounds and compositions, as the name implies, are useful for modulating or regulating immunological functions in animals. Immunomodulators may be immunostimulants for building up immunities to, or initiate healing from, certain diseases and disorders. Conversely, immunomodulators may be immunoinhibitors or immunosuppressors for preventing undesirable immune reactions of the body to foreign materials, or to prevent or ameliorate autoimmune reactions or diseases.

Immunomodulators have been found to be useful for treating systemic autoimmune diseases, such as lupus erythematosus and diabetes, as well as immunodeficiency diseases. Further, immunomodulators may be useful for immunotherapy of cancer or to prevent rejections of foreign organs or other tissues in transplants, e.g., kidney, heart, or bone marrow.

Usually, an infectious disease is a disease which arises when a malfunction of the immune system (a self-defense system for protecting the living body (host) from infections by exogenous pathogens, such as viruses, bacteria, parasites and fungi) occurs, so that the exogenous pathogens cannot be excluded from the living body. On the other hand, an autoimmune disease is a disease in which the immune system malfunctions to attack the “self” (which should not be attacked but should be defended by the immune system). Two types of autoimmune diseases, namely the autoimmune diseases specific to an organ or tissue and the non-specific systemic autoimmune diseases, are known in the art. A wide variety of autoimmune diseases are known to result from immunomodulation disorders, and examples of such autoimmune diseases include systemic lupus erythematosus, rheumatoid arthritis, type I diabetes, inflammatory bowel disease, biliary cirrhosis, uveitis, multiple sclerosis, Crohn's disease, ulcerative colitis, bullous pemphigoid, sarcoidosis, psoriasis, ichthyosis and Graves' orbitopathy.

Accordingly, the present invention further provides a method for the treatment of diseases caused or affected by malfunction of the immune system comprises administering the composition of the invention, to a human or animal in need of such treatment.

In the treatment method of the invention, the immune diseases are infections by exogenous pathogens (such as viruses, bacteria, parasites or fungi), autoimmune diseases (such as systemic lupus erythematosus, rheumatoid arthritis, type I diabetes, inflammatory bowel disease, biliary cirrhosis, uveitis, multiple sclerosis, Crohn's disease, ulcerative colitis, bullous pemphigoid, sarcoidosis, psoriasis, ichthyosis or Graves' orbitopathy). The present invention can be applied for cancer immunotherapy.

One trained in the art can readily formulate the present composition into any of these convenient forms for oral or topical administration.

### EXAMPLES

Mice and Serum Samples

Sixty female Balb/c mice, purchased from the National Cheng Kung University (Tainan, Taiwan) at four weeks of age and weighing between 14 and 16 gms, were used for this study. Yi-fey Ruenn-hou (YR) Tea consisted of 10% licorice root, 10% American ginseng, 10% white peony root and 70% green tea. The origin source of licorice root, American ginseng, and white peony root is from China. The green tea is from Taiwan. YR Tea was prepared by soaking the four combined herbs at 100° C. water for 1 hour and left to cool off at room temperature. The mice were randomly divided into four groups (15 mice/group) with 1 ml drinking water (group A) or 2 mg/ml (group B), 8 mg/ml (group C), 40 mg/ml (group D) of YR Tea administered daily. Since YR Tea has been used for centuries in the traditional Chinese medicine, the long term effects on toxicity and cytokine modulation were aimed to be investigated in this study. A minimum of two mice were sacrificed at the end of each month for six months after the regimen commenced. Sera were separated from the blood samples of sacrificed mice by centrifugation at 3,500×g for 15 min and aliquoted and stored at −70° C. until required for cytokine assay. The brain, heart and liver were removed, and placed in 10% formalin for hematoxylin-eosin (H&E) staining.

Hematoxylin-Eosin (H&E) Staining

All specimens were examined by routine fixation with H&E staining. All brains, hearts livers, spleens and kidneys were fixed immediately in a 10% formalin solution overnight at 4° C. and embedded in paraffin. Serial sagittal sections 5 μm thick were cut, and stained with H&E for histological study.

Cytokines Determinations by ELISA

The cytokines (IL-2, IL-4, IL-10 and IFN-γ) were assayed in triplicate using commercial ELISA kits (Pharmingen, San Diego, Calif. USA) according to manufacturer's instructions. Using 96-well plates, 100 μl of 1:250 diluted anti-mouse capture antibody sera were added and incubated overnight at 25° C. Volumes of 100 μl of 1:4 diluted sera in assay diluents or standard were added and plates were incubated at 25° C. for two hours. After incubation, plates were washed five times with ELISA washing solution. Then, 100 μl of working detector (biotinylated anti-mouse monoclonal antibody conjugated to horseradish peroxidase at 1:250 dilution in assay diluents) were added. Plates were incubated at room temperature for one hour. After incubation, the wells were washed seven additional times with ELISA washing solution using an ELISA washer (BIO-RAD, Hercules, Calif., USA). 100 μl substrate solution (Tetramethylbenzidine) were added and plates were incubated at 37° C. After incubation for 15 min, the reaction was terminated by the stop solution (2N H₂SO₄) and the absorbance was read at 450 nm (Spectrophotometer Model 550, BIO-RAD). For every test, a standard curve was also derived for IL-2, IL-4, IL-10 and IFN-γ. The range of the standard curve was extended as 0.0000 pg to 1000 pg for IL-2, 0.0000 pg to 1000 pg for IL-4, 0.0000 pg to 4000 pg for IL-10, and 0.0000 pg to 8000 pg for IFN-γ.

Statistical Analyses

The Kruskal-Wallis test was used to test the differences on weight among different mice of various groups and P values <0.05 were taken to be significant. The Wilcoxson rank sum test was used to test the weight difference and cytokine ratio between each two different groups and P values <0.05 were taken to be significant.

Results

At the end of each month during the study period, two or more mice from each group were sacrificed. The weight (Table 1) and physiological characteristics of the mice were recorded for each group. The results indicated that no weigh difference existed among the various groups (P>0.05). There was no pathological change examined by H&E staining in the brain, the heart, the liver, the spleen and the kidneys of the mice given YR Tea (histology data not shown).

TABLE 1
The mean weight (gms) ± standard deviation of mice fed with
0 mg/ml (group A), 2 mg/ml (group B), 8 mg/ml (group C),
or 40 mg/ml (group D) of YR Tea.

| | Mean weight (gms) ± standard deviation | | | |
|---|---|---|---|---|
| Month | Group A | Group B | Group C | Group D |
| 1 | 31.87 ± 2.29 | 31.12 ± 3.04 | 31.98 ± 2.48 | 32.40 ± 2.29 |
| 2 | 29.95 ± 2.82 | 33.56 ± 2.23 | 32.44 ± 2.20 | 30.85 ± 2.03 |
| 3 | 34.29 ± 3.60 | 30.90 ± 1.51 | 33.00 ± 2.08 | 35.26 ± 2.70 |
| 4 | 33.90 ± 2.58 | 34.50 ± 2.42 | 33.80 ± 2.51 | 34.73 ± 2.67 |
| 5 | 32.80 ± 2.20 | 32.10 ± 2.86 | 33.20 ± 1.86 | 39.70 ± 8.94 |
| 6 | 36.40 ± 1.66 | 34.10 ± 4.75 | 35.24 ± 2.74 | 35.84 ± 2.95 |

No mice in any group died or wounded during the experimental period. The serum sample from each mouse was collected separately for IL-2, IL-4, IL-10 and IFN-γ ELISA assay. Standard curves for the cytokines were also established and covered a wide range. The cytokine concentrations (pg/ml) of mice fed with 0 mg/ml (group A), 2 mg/ml (group B), 8 mg/ml (group C), or 40 mg/ml (group D) of YR Tea from months 1 to 6 were identified. The cytokines concentration for the control group ranged from 0.0346 to 11.8527 pg/ml for IL-2, 0.0114 to 1.7040 pg/ml for IL-4, 0.0116 to 0.1934 pg/ml for IL-10, and 0.0646 to 10.6615 pg/ml for IFN-γ. Compared with the control group, the cytokine concentrations in the mice fed with YR Tea showed a greater degree of variation. When the mice were given 2 mg/ml of YR Tea, IL-2 varied from 0.0358 to 22.8441 pg/ml, IL-4 from 0.0120 to 5.1504 pg/ml, IL-10 from 0.0109 to 4.0254 pg/ml and IFN-γ from 0.0606 to 21.4649 pg/ml. When the mice were given 8 mg/ml YR Tea, IL-2 varied from 0.0402 to 13.4653 pg/ml, IL-4 from 0.0107 to 10.0454 pg/ml, IL-10 from 0.0110 to 2.9018 pg/ml and IFN-γ from 0.0636 to 16.2793 pg/ml. When the mice were given 40 mg/ml YR Tea, IL-2 varied from 0.0349 to 14.3708 pg/ml, IL-4 from 0.0129 to 31.1712 pg/ml, IL-10 from 0.0120 to 18.0052 pg/ml and IFN-γ from 0.0639 to 79.2925 pg/ml. The mean cytokine concentrations of mice given water (group A), 2 mg/ml (group B), 8 mg/ml (group C), or 40 mg/ml (group D) of YR Tea were listed in Table 2.

TABLE 2
The mean cytokine concentrations ± standard deviation of mice given water
(group A), 2 mg/ml (group B), 8 mg/ml (group C), or 40 mg/ml (group D) of YR Tea.

Month

| Cytokine | Group | 1 | 2 | 3 | 4 | 5 | 6 |
|---|---|---|---|---|---|---|---|
| IL-2 | A | 7.6136 ± 4.6022 | 3.1528 ± 1.7531 | 2.0344 ± 1.0326 | 1.4047 ± 0.2385 | 1.1505 ± 0.0248 | 1.1624 ± 0.0638 |
| | B | 9.3647 ± 2.8121 | 3.2789 ± 1.0246 | 2.1565 ± 0.7019 | 1.4890 ± 0.4350 | 1.2771 ± 0.2180 | 2.4062 ± 0.6501 |
| | C | 8.6034 ± 3.1276 | 3.1843 ± 0.9243 | 2.0547 ± 0.8362 | 1.5592 ± 0.6037 | 1.2195 ± 0.0352 | 1.7204 ± 0.3421 |
| | D | 10.5068 ± 2.3074 | 3.2790 ± 0.4731 | 4.2926 ± 1.5017 | 1.8402 ± 0.5212 | 1.9098 ± 0.7025 | 1.3484 ± 0.5102 |
| IL-4 | A | 0.3623 ± 0.0550 | 1.4728 ± 0.6548 | 0.2048 ± 0.1037 | 0.1995 ± 0.0474 | 0.1074 ± 0.0821 | 0.1148 ± 0.0903 |
| | B | 0.4239 ± 0.0620 | 1.6790 ± 0.1304 | 0.4485 ± 0.0276 | 0.3451 ± 0.0114 | 0.1557 ± 0.0089 | 0.3272 ± 0.0116 |
| | C | 0.6376 ± 0.2062 | 1.5170 ± 0.3754 | 0.5989 ± 0.0167 | 0.2175 ± 0.0470 | 0.7454 ± 0.0097 | 0.1619 ± 0.0134 |
| | D | 0.3804 ± 0.0091 | 1.9294 ± 0.2035 | 20.8404 ± 6.0274 | 0.3411 ± 0.0687 | 0.2481 ± 0.0107 | 0.2491 ± 0.0194 |
| IL-10 | A | 0.0387 ± 0.0112 | 0.1842 ± 0.0518 | 0.0247 ± 0.0098 | 0.0234 ± 0.0125 | 0.0324 ± 0.0128 | 0.0257 ± 0.0138 |
| | B | 0.0379 ± 0.0077 | 0.1989 ± 0.1073 | 0.0262 ± 0.0064 | 0.0335 ± 0.0068 | 0.0697 ± 0.0147 | 1.8278 ± 0.3012 |
| | C | 0.0480 ± 0.0064 | 0.1860 ± 0.0207 | 0.0257 ± 0.0093 | 0.0274 ± 0.0026 | 0.0415 ± 0.0028 | 1.3161 ± 0.5212 |
| | D | 0.0430 ± 0.0087 | 0.1953 ± 0.0760 | 0.1040 ± 0.0540 | 0.0471 ± 0.0079 | 11.4479 ± 3.7801 | 0.5042 ± 0.0243 |
| IFN-γ | A | 1.7838 ± 0.0184 | 9.6627 ± 3.3985 | 2.0658 ± 1.0628 | 3.8973 ± 1.0847 | 1.3204 ± 0.8616 | 1.8827 ± 0.9621 |
| | B | 1.8373 ± 0.3649 | 11.2087 ± 3.6401 | 3.8630 ± 0.8032 | 6.8982 ± 1.5548 | 1.9938 ± 0.1961 | 5.0268 ± 1.7949 |
| | C | 2.4260 ± 0.3016 | 9.7593 ± 3.7081 | 2.3344 ± 0.6068 | 4.4039 ± 1.0062 | 2.1787 ± 0.4976 | 2.3157 ± 0.8102 |
| | D | 1.8195 ± 0.7451 | 10.8222 ± 3.1102 | 73.2946 ± 22.8767 | 4.0142 ± 1.6104 | 1.3468 ± 0.3408 | 1.9015 ± 0.2460 |

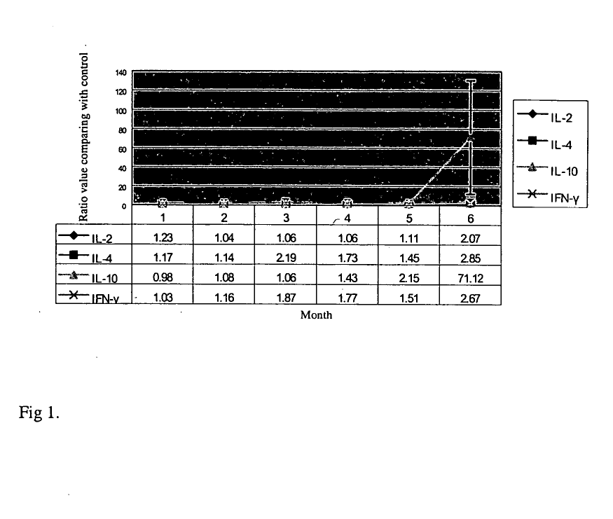

In order to evaluate the cytokine variation as ratios, the cytokine levels in the YR tea treated mice were compared with the control (group A) values. FIGS. 1˜3 compare the varied cytokine ratio for mice given 2 mg/ml, 8 mg/ml or 40 mg/ml of YR Tea with those in the control groups. The Wilcoxson rank sum test was used to test the cytokine ratio between each two different groups. For group B, cytokines IL-2, IL-4, IL-10 and IFN-γ did not vary markedly during the first five months. Their concentration ratio was between 0.98˜2.19 compared with group A, however, the concentration ratio of IL-10 increased significantly in month six at 71.12 (P<0.05). For group C, the concentration ratio for all cytokines was not varied markedly during the first four months. It was between 1.01 to 1.76 compared with group A. But for IL-4, it peaked at 6.94 in month five. For IL-10, it peaked at 51.21 in month six. For IL-2 and IFN -γ, the variation in cytokine concentration was not obvious, with a ratio between 1.01 to 1.65. For group D, the concentration of IL-4 and IFN-γ increased significantly in month three where the ratio peaked at 101.76 and 35.48 respectively (P<0.05). The concentration ratio of IL-10 peaked at 353.33 in month five. For IL-2, there was no apparent variation in concentration ratio, which was between 1.01 to 1.48.

Discussion

All components, licorice root, American ginseng, Radix Paeoniae alba and green tea, which were combined into YR Tea used in our study, have direct or indirect effects on immunomodulation. As licorice root and green tea, many herbs contain potent antioxidant compounds that provide significant protection against chronic diseases or have antiviral or antitumor activity (Craig, W. J. Health-promoting properties of common herbs. Am J Clin Nutr 1999, 70: 491S-499S). Kroes et al. (Kroes, B. H.; Beukelman, C. J.; van den Berg, A. J.; Wolbink, G. J.; van Dijk, H.; Labadie, R. P. Inhibition of human complement by beta-glycyrrhetinic acid. Immunology 1997, 90: 115-120) found that beta-glycyrrhetinic acid is a potent inhibitor of the classical complement pathway, whereas no inhibitory activity was observed against the alternative pathway and beta-glycyrrhetinic acid acts at the level of complement component C2. The hepatoprotective effect of licorice root has recently been explained as the inhibitory effects on immune-mediated cytotoxicity against hepatocytes and on nuclear factor (NF)-kappa B, which activates genes encoding inflammatory cytokines in the liver (Shibata, S. A drug over the millennia: pharmacognosy, chemistry, and pharmacology of licorice. Yakugaku Zasshi 2000, 120: 849-862). Tomada et al. (Tomoda, M.; Matsumoto, K.; Shimizu, N.; Gonda, R.; Ohara, N.; Hirabayashi, K. An acidic polysaccharide with immunological activities from the root of Paeonia lactiflora. Biol Pharm Bull 1994, 17: 1161-1164) demonstrated that an acidic polysaccharide, which was isolated from the root of Paeonia lactiflora pallas exhibited remarkable reticuloendothelial system-potentiating activity in a carbon clearance test and considerable anti-complementary activity. As nitric oxide plays an important role in immune function, Friedl et al. (Friedl, R.; Moeslinger, T.; Kopp, B.; Spieckermann, P. G. Stimulation of nitric oxide synthesis by the aqueous extract of Panax ginseng root in RAW 264.7 cells. Br J Pharmacol 2001, 134: 1663-1667) revealed that Panax ginseng treatment could modulate several aspects of host defense mechanisms due to stimulation of inducible nitric oxide synthase. Singh et al. (Singh, R.; Ahmed, S.; Malemud, C. J.; Goldberg, V. M.; Haqqi, T. M. Epigallocatechin-3-gallate selectively inhibits interleukin-1beta-induced activation of mitogen activated protein kinase subgroup c-Jun N-terminal kinase in human osteoarthritis chondrocytes. J Orthop Res 2003, 21: 102-109) reported that epigallocatechin-3-gallate (EGCG), a green tea polyphenol might be of potential benefit in inhibiting IL-1beta-induced catabolic effects in osteoarthritis chondrocytes that are dependent on JNK activity.

A network of cytokines regulates the growth and function of the cells of the immune system. T cells possess a dominant role in this network since they are the main source of many cytokines. The production of different cytokines is specifically regulated by means of cell interactions and cytokine concentration and is depend largely on the state of differentiation of the T cell. Two different types of differentiated T cells can be characterized according to the pattern of cytokine production of T cells: IL2 and IFN-□ are typically produced by T helper 1 (TH1) cells, whereas predominantly T helper 2 (TH2) cells produce ILA, IL5 and IL10. TH1 and TH2 cytokines exert a mutual cross-regulation on the precursors of TH1- or TH2-type effector cells which are important mediators in directing the immune system towards the appropriate response. In vivo and in vitro studies have demonstrated the selective regulatory effect that TH1 and TH2 cytokines reciprocally exert in the regulation of the polarization of precursor cells into TH1 or TH2 types. The selective activation of either TH 1 or TH 2 type cells depends on the antigen and is influenced by cytokines produced partly by antigen-presenting cells and partly by T helper cells (Schwarz, M.; Sunder-Plassmann, R.; Cerwenka, A.; Pickl, W. F.; Holter, W. Regulation of cytokine production by human T-lymphocytes in allergic immune response. Wien Klin Wochenschr 1993, 105: 672-676). Recently, it was suggested that NF-kappaB may be one of multiple factors in the immunosuppressive effects mediated by tea pigments (Tomita, M.; Irwin, K. I.; Xie, Z. J.; Santoro, T. J. Tea pigments inhibit the production of type 1 (T(H1)) and type 2 (T(H2)) helper T cell cytokines in CD4(+) T cells. Phytotherapy Research 2002, 16: 36-42).

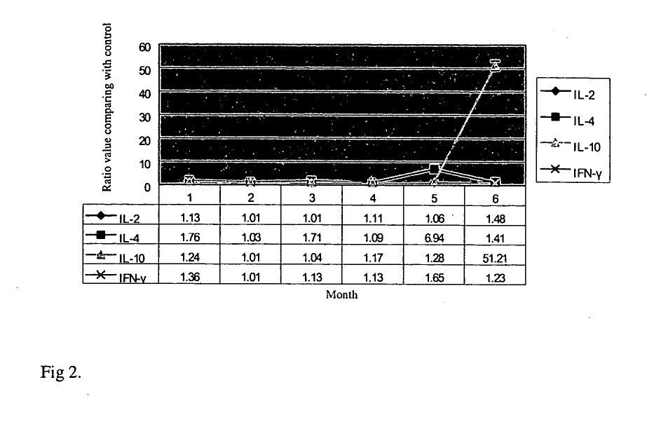

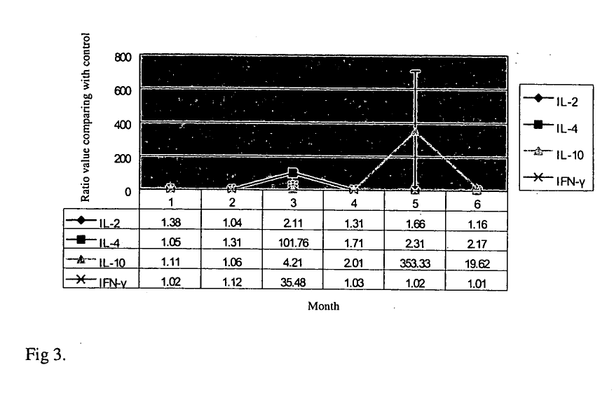

In the experiment of the invention, it demonstrated that YR Tea has different immunomodulation effects on TH1 cytokines, i.e. IL-2 or IFN-γ and TH2 cytokines, i.e. IL-4 or lL-10. As the results demonstrated in FIGS. 1 to 3, IL-2 and EFN-γ, TH1 cytokines, have no obvious variation in group B and group C. Group B was stimulated for the secretion of IL-10 which occurred in month six, and for group C, the serum IL-4 increased markedly and reached the maximum in month five, and the serum IL-10 concentration reached the maximum in month six. Group D maxima for the TH2-pattern cytokines IL-4 and IL-10 were demonstrated in months three and five respectively. Group D was also stimulated for the secretion of the TH1-pattern cytokine IFN-γ, reaching maximum in month three. These results suggested that TH2-pattern cytokines responded earlier and higher in group D than in groups B and C. The higher concentration of IFN-γ detected in group D might be induced by TH2-pattern cytokines through the cytokine network system to balance TH1/TH2 immune response. Comparing groups B, C, and D, the cytokine modulation effects are consistent, and the most obvious cytokine modulation effect was observed in group D, which was the highest dosage employed for treating the mice. In addition, there was no side effects and toxicity in mice given YR Tea in all groups since no significant weight difference nor pathological changes were observed. These data suggested that increased secretion of both TH1- and TH2-pattern cytokines in mice serum could be achieved with a dietary combined herb-soaked solution. The effect of YR Tea on cytokine modulation in vivo is predominantly TH2-pattern and is dependent on its dosage (P<0.05).

1. A composition for use in immunomodulation comprising 1 to 30 weight percent of licorice root, 1 to 30 weight percent of American ginseng, 1 to 30 weight percent of Radix Paeoniae alba and 3 to 97 weight percent of green tea (Camellia sinensis).

2. The composition according to claim 1, wherein the concentration of licorice root ranges from 1 to 25 weight percent.

3. The composition according to claim 1, wherein the concentration of American ginseng ranges from 1 to 25 weight percent.

4. The composition according to claim 1, wherein the concentration of Radix Paeoniae alba ranges from 1 to 25 weight percent.

5. The composition according to claim 1, wherein the concentration of green tea ranges from 40 to 95 weight percent.

6. The composition according to claim 2, wherein the concentration of licorice root is 10 weight percent.

7. The composition according to claim 3, wherein the concentration of American ginseng is 10 weight percent.

8. The composition according to claim 4, wherein the concentration of Radix Paeoniae alba is 10 weight percent.

9. The composition according to claim 5, wherein the concentration of green tea is 50-80 weight percent.

10. The composition according to claim 5, wherein the concentration of green tea is 70 weight percent.

11. The composition according to claim 1 wherein the licorice root is in the form of licorice root extract.

12. The composition according to claim 1 wherein the American ginseng is in the form of American ginseng extract.

13. The composition according to claim 1 wherein the Radix Paeoniae alba is in the form of Radix Paeoniae alba extract.

14. The composition according to claim 1 wherein the green tea is in the form of green tea extract.

15. A method for the treatment of diseases caused or affected by malfunction of immune system comprises administering the composition according to claim 1, to a human or animal in need of such treatment.

16. The method according to claim 15, wherein the diseases are infections by exogenous pathogens

17. The method according to claim 16, wherein the exogenous pathogens are viruses, bacteria, parasites or fungi.

18. The method according to claim 15, wherein the diseases are autoimmune diseases.

19. The method according to claim 18, wherein the autoimmune diseases are systemic lupus erythematosus, rheumatoid arthritis, type I diabetes, inflammatory bowel disease, biliary cirrhosis, uveitis, multiple sclerosis, Crohn's disease, ulcerative colitis, bullous pemphigoid, sarcoidosis, psoriasis, ichthyosis or Graves' orbitopathy.

20. The method according to claim 15, which can be applied for cancer immunotherapy.
